(19) United States
(12) Patent Application Publication
(10) Pub. No.: US 2007/0027495 A1
(43) Pub. Date: Feb. 1, 2007

(54) External bladder sensor for sensing bladder condition

(75) Inventor: Martin T. Gerber, Maple Grove, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/194,076

(22) Filed: Jul. 29, 2005

(51) Int. Cl. A61N 1/00

(52) U.S. Cl. 607/41

(57) ABSTRACT

The disclosure describes an implantable bladder sensor that is attachable to an exterior surface of a urinary bladder to sense bladder condition or activity. The sensor may include a strain gauge that detects mechanical deformation of the bladder. Mechanical deformation may be indicative of a gradual filling of the bladder, or an instantaneous contraction indicating an imminent urine voiding event. Wireless telemetry circuitry within the sensor transmits information to implanted electrical stimulator that delivers electrical stimulation for alleviating urinary incontinence, or to an external programmer that controls the implanted stimulator.

### TECHNICAL FIELD

The invention relates to implantable medical devices and, more particularly, implantable sensors.

### BACKGROUND

Urinary incontinence, or an inability to control urinary function, is a common problem afflicting people of all ages, genders, and races. Various muscles, nerves, organs and conduits within the urinary tract cooperate to collect, store and release urine. A variety of disorders may compromise urinary tract performance and contribute to incontinence. Many of the disorders may be associated with aging, injury or illness.

In some cases, urinary incontinence can be attributed to improper sphincter function, either in the internal urinary sphincter or external urinary sphincter. For example, aging can often result in weakened sphincter muscles, which causes incontinence. Some patients also may suffer from nerve disorders that prevent proper triggering and operation of the bladder or sphincter muscles. Nerves running though the pelvic floor stimulate contractility in the sphincter. A breakdown in communication between the nervous system and the urinary sphincter can result in urinary incontinence.

Electrical stimulation of nerves in the pelvic floor may provide an effective therapy for a variety of disorders, including urinary incontinence. For example, an implantable electrical stimulator may be provided. The electrical stimulator may be a neurostimulator that delivers electrical stimulation to the sacral nerve to induce sphincter constriction and thereby close or maintain closure of the urethra at the bladder neck. In addition, electrical stimulation of the bladder wall may enhance pelvic floor muscle tone and assist fluid retention in the bladder or voiding fluid from the bladder. An appropriate course of neurostimulation therapy may be aided by a sensor that monitors physiological conditions of the bladder. In some cases, an implantable stimulation device may deliver stimulation therapy based on the level or state of a sensed physiological condition.

### SUMMARY

The invention is directed to a sensor that is implantable to sense bladder condition. The sensor is configured for placement on an exterior wall of the bladder, and may sense a change in bladder fill stage, which may be due to the adding or voiding of urine, or a bladder contraction, which may signal an imminent voiding event. The invention also contemplates a neurostimulation system and method that make use of such a sensor for alleviation of urinary incontinence. The sensor includes a sensing component, such as a strain gauge to detect the level or degree of stretch of the bladder wall. In this manner, the sensor can sense bladder wall distension, contraction, or both. The bladder sensor may be able to detect the fill stage or contraction of the bladder at any given time, and provide signals indicating such conditions to an implanted neurostimulator, an external programmer, or a data recorder.

Inadequate sphincter force, insufficient pelvic floor muscle tone, other pelvic floor disorders, or neurological disorders may result in involuntary bladder voiding, i.e., incontinence. The bladder sensor may provide short- or long-term monitoring of bladder stretch or size, e.g., for analysis by a clinician. Alternatively, a bladder sensor may form part of a closed-loop neurostimulation system. For example, neurostimulation therapy can be applied to increase pelvic floor muscle tone, urinary sphincter pressure, or both, and thereby prevent involuntary urine leakage. In particular, an implantable neurostimulator may be responsive to a bladder condition representative of a bladder fill stage or bladder contraction. Neurostimulation techniques may increase stimulation to prevent or stop voiding when voiding is not desired by the patient, thus alleviating urinary incontinence.

In one embodiment, the invention provides a method comprising sensing bladder condition via a implanted sensor attached to an exterior surface of a bladder and generating information based on the sensed bladder condition.

In another embodiment, the invention provides an implantable electrical stimulation system comprising an implantable sensor that senses condition of a bladder, a fixation structure that attaches the implantable sensor to an exterior surface of the bladder, and processing circuitry that generates information based on the sensed bladder condition.

In an additional embodiment, the invention provides an implantable medical device comprising a sensor housing, a sensing element, associated with the housing, that senses condition of a bladder, a fixation structure that fixes the sensor housing to an exterior surface of the bladder, and processing circuitry that generates information based upon the sensed bladder condition.

In various embodiments, the invention may provide one or more advantages. For example, measuring bladder condition with a sensor at an exterior wall of the bladder provides direct contact with the bladder wall while reducing the possibility of urinary infections that could otherwise occur due to sensor presence within the interior of the bladder. Mounting the sensor at the exterior bladder wall may relax sensor size limitations relative to sensors introduced into the bladder or urethra. The bladder sensor permits bladder condition measurements to be taken periodically or continuously and saved to memory, either within the sensor, an implantable neurostimulator or an external programmer.

The bladder condition measurements may also be sent via wireless telemetry to an implantable stimulator to trigger or control delivery of therapy for any detected urinary incontinence events. In addition, in some embodiments, the bladder sensor may notify the patient of a filled bladder and urge the patient to urinate before causing an unintentional voiding event. Also, with closed-loop stimulation, a stimulator may generate stimulation parameter adjustments, based on the sensed conditions, to more effectively target the function of the urinary sphincter muscle or pelvic floor muscle tone, thereby enhancing stimulation efficacy. In some patients, more effective stimulation via the sacral nerve may actually serve to strengthen the sphincter muscle or enhance pelvic floor tone, restoring proper operation.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an implantable stimulation system, incorporating an implantable bladder sensor, for treating urinary incontinence.

FIG. 2 is a cross-sectional side view of an implantable bladder sensor attached to the exterior wall of the bladder of a patient.

FIG. 3 is a bottom view of the implantable bladder sensor of FIG. 2.

FIG. 4 is a cross-sectional side view of a deployment device during deployment and fixation of the implantable bladder sensor of FIG. 2

FIG. 5 is an enlarged schematic diagram illustrating an implantable bladder sensor sutured to the outside of the bladder of a patient.

FIG. 6 is an enlarged, bottom view of the implantable bladder sensor of FIG. 5.

FIG. 7 is a cross-sectional side view of an implantable bladder sensor placed within the bladder wall of a patient.

FIG. 8 is a schematic diagram illustrating endoscopic deployment of the implantable bladder sensor of FIG. 7.

FIG. 9 is a functional block diagram illustrating various components of an exemplary implantable bladder sensor.

FIG. 10 is a functional block diagram illustrating various components of an implantable stimulator that communicates wirelessly with an implantable bladder sensor.

FIG. 11 is a flow chart illustrating a technique for delivery of stimulation therapy based on closed loop feedback from an implantable bladder sensor.

FIG. 12 is a flow chart illustrating an alternative technique for delivery of stimulation therapy based on closed loop feedback from an implantable bladder sensor.

### DETAILED DESCRIPTION

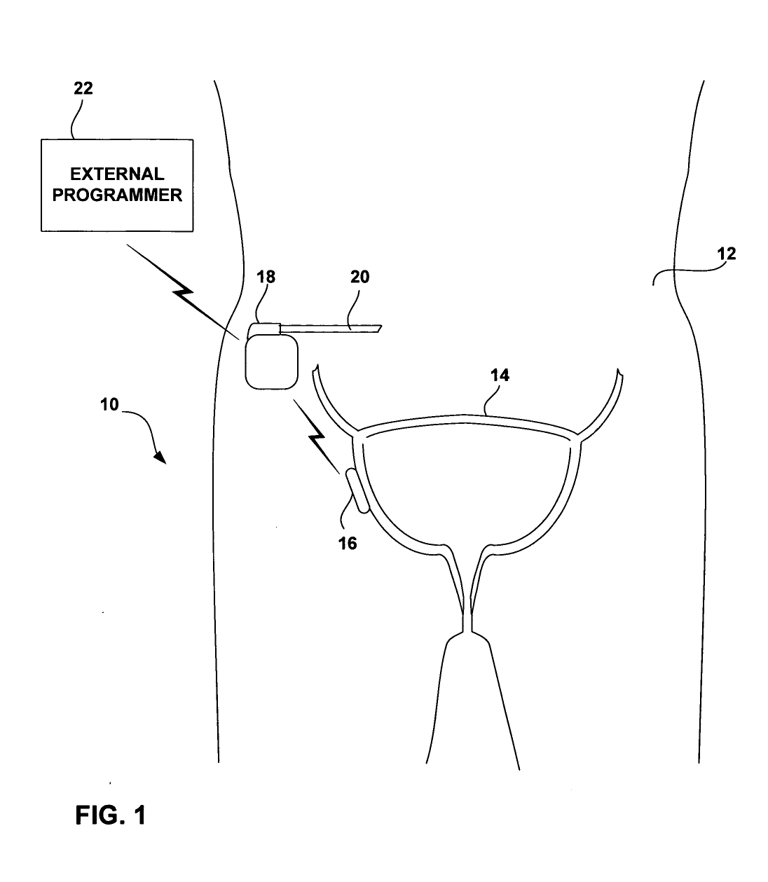

FIG. 1 is a schematic diagram illustrating an implantable stimulation system 10 for alleviation of urinary incontinence. As shown in FIG. 1, system 10 may include an implantable bladder sensor 16, implantable stimulator 18 and external programmer 22 shown in conjunction with a patient 12. Bladder sensor 16 may sense bladder condition in terms of changes in bladder size, bladder wall thickness, shape, volume or deformation in the wall of bladder 14. Deformation of bladder 14 is detected by a sensing element such as strain gauge, for example, in bladder sensor 16 to generate information regarding the amount of urine in bladder 14, i.e., a fill stage, or the occurrence of bladder contraction above a threshold. A fill stage or bladder contraction may be considered bladder activity or bladder condition information, as described herein. Stimulator 18 may activate or adjust stimulation in response to a fill stage or contraction sensed by bladder sensor 16.

Bladder sensor 16 transmits the sensed bladder condition information to at least one of stimulator 18 or external programmer 22 by wireless telemetry. The information may be transmitted as individual measurement samples, or pre-processed bladder condition information based on one or more measurement samples. In some embodiments, the information may be transmitted only when a significant change is detected. Simulator 18 or programmer 22 may record the information, generate adjustments to electrical stimulation parameters applied by the stimulator, or both. In some embodiments, bladder sensor 16 may support purely diagnostic purposes, such as urodynamic study, e.g., by transmission of information to external programmer 22. In other embodiments, bladder sensor 16 may form part of a closed loop feedback system for delivery of neurostimulation therapy by stimulator 18 to patient.

Implantable stimulator 18 is coupled to lead 20, which is tunneled through patient 12 to one or more nerve sites. Lead 20 contains one or more electrodes at the distal end to transfer electrical stimulation from stimulator 18 to nerves which innervate the urinary system. Lead 20 may terminate adjacent nerves in the pelvic floor, such as the sacral nerve or pudendal nerve. For example, sacral nerve stimulation may result in an increase in pelvic floor muscle tone or the contraction of the urinary sphincter, which keeps urine inside bladder 14. Appropriate nerve stimulation may assist patient 12 in avoiding urinary incontinence or promoting the elimination of urine from bladder 14.

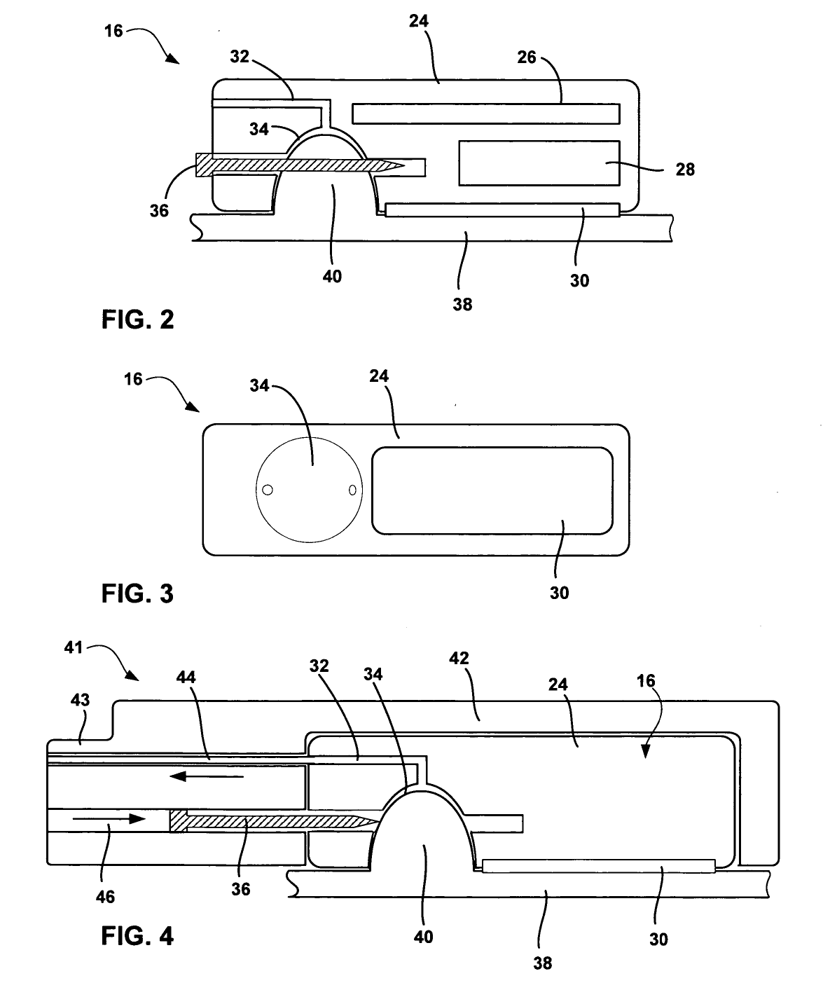

FIG. 2 is a cross-sectional illustration of an implantable bladder sensor attached to the outside of the bladder of a patient. As shown in FIG. 2, bladder sensor 16 includes a sensor housing 24 and sensing element 30 that extends from the housing. Sensing element 30 may be a strain gauge sensor that senses mechanical deformation of the wall of bladder 14. Sensing element 30 may be coupled to a circuit board 26 within bladder sensor 16. A power source 28, such as a battery, may be provided to power circuit board 26, sensing element 30 or both. Circuit board 26 includes processing electronics to process signals generated by sensing element 30, and generate bladder information based on the signals. In addition, circuit board 26 may include telemetry circuitry for wireless telemetry with stimulator 18, external programmer 22, or both. Bladder sensor 16 is attached to bladder wall 38 by fastening pin 36 through tissue 40. Vacuum channel 32 applies negative pressure in vacuum cavity 34 to draw in a portion of bladder wall 38, i.e., tissue 40.

Power source 28 may take the form of a small rechargeable or non-rechargeable battery, which may be configured as a coin cell or pin cell. Different types of batteries or different battery sizes may be used, depending on the requirements of a given application. To promote longevity, power source 28 may be rechargeable via induction or ultrasonic energy transmission, and includes an appropriate circuit for recovering transcutaneously received energy. For example, power source 28 may include a secondary coil and a rectifier circuit for inductive energy transfer. Power generation or charging electronics may be carried on circuit board 26. In still other embodiments, power source 28 may not include any storage element, and sensor 16 may be fully powered via transcutaneous inductive energy transfer. As a further alternative, stimulator 18 or programmer 22 may be configured to apply inductive power to sensor 16 whenever sensing is desired. In this case, when inductive power is not applied, sensor 16 is asleep. Upon application of inductive power, sensor 16 wakes up, acquires a sense signal, and transmits the signal to programmer 22 or stimulator 18. Accordingly, stimulator 18 or programmer 22 determine the sampling rate of sensor 16 by powering up the sensor at desired intervals.

In the exemplary embodiment of FIG. 2, bladder sensor 16 includes a strain gauge as sensing element 30 to sense mechanical deformation of the wall of bladder 14 and thereby indicate changes in bladder 14 size or shape or sense contractions. Sensing element 30 senses the stretch of bladder 14 to detect the expansion and contraction, or increase and decrease, in size of bladder 14, and thereby senses a fill stage of the bladder. The expansion and contraction may be monitored as gradual or instantaneous changes. For example, gradual expansion may indicate a gradual filling of bladder 14, while a rapid or instantaneous change may indicate a bladder muscle contraction and the possibility of imminent, involuntary voiding.

The disclosure is not limited to the use of a strain gauge for sensing or detecting changes in the size, wall thickness, shape or volume of bladder 14. For example, other embodiments may include one or more electrodes for sensing the electrical activity of the muscles surrounding bladder 14. Detecting muscle activity may be correlated with changes in bladder size or contraction. In other embodiments, bladder sensor 16 may utilize an ultrasound transducer to sense the thickness of the wall of bladder 14 or the distance to the opposite wall of bladder 14. Further, bladder sensor 16 may contain more than one sensing component, such as two strain gauges. In each case, bladder sensor 16 is deployed on or within an exterior wall of bladder 14.

Strain gauge sensing element 30 may be formed with a flexible material, including polyurethane or silicone. In other embodiments, the strain gauge may be formed with a flexible polymer or metal alloy. The strain gauge may be able to sense small changes in bladder 14 wall stretch or deformation for detection of bladder filling. The strain gauge may carry a circuit containing resistive elements, which may be printed, deposited or otherwise formed on the flexible material. In some embodiments, the strain gauge may include small protrusions or adhesion points with stick to certain locations on bladder wall 38. As bladder wall 38 expands or contracts, these locations will move with respect to each other.

Strain gauge sensing element 30 senses the movement of bladder wall 38 in terms of changes in impedance, voltage, or other electrical characteristics of the circuit formed on the strain gauge to sense the expansion or contraction of bladder 14. Processing electronics carried by circuit board 26, or carried by stimulator 18 or external programmer, process the sensed bladder condition or activity signal to detect expansion or contraction of the bladder 14. In particular, the signal output by sensing element 30 can be used to sense a urine fill stage of bladder 14, which may be indicative of progression toward a voiding event, or a muscle contraction, which may be indicative of an imminent voiding event.

The electrical characteristics may be monitored for rapid or instantaneous changes indicative of bladder contraction, as well as slow, gradual changes indicative of bladder filling. Rapid and gradual changes may both indicate progression of the bladder toward an imminent voiding event. For example, contraction may result in an immediate leakage of urine, while bladder filling may result in an eventual leakage of urine when the bladder becomes too full. In both cases, activation or adjustment of electrical stimulation may be desirable to prevent involuntary leakage. The characteristics measured by sensing element 30 and processing electronics carried by circuit board 26 may be sent to stimulator 18 or programmer 32 as raw measurements or as bladder condition or activity signals indicating a bladder condition, such as a state of fullness or a contractile condition.

As described herein, sensing element 30 may be configured to sense both fill stage and instantaneous contractions. For example, sensing element 30 may sense bladder wall deformation to sense slow changes in the size of bladder 14. The amount of bladder wall deformation may correlate with a fill stage. As the fill stage increases, stimulator 18 may apply progressively greater levels of stimulation to prevent an involuntary voiding event, i.e., unintended bladder leakage. Accordingly, stimulator 18, either independently or under control of programmer 22, may adjust the stimulation level as the amount of bladder wall deformation sensed by sensor 16 indicates a particular fill stage, and may adjust the stimulation level in steps over a series of sensed fill stages. The stimulation level may be adjusted in discrete steps or in proportion to the sensed fill stage.

In addition to sensing gradually changing deformation levels indicative of fill stage, sensing element 30 also may sense rapid or instantaneously changing deformation levels indicative of bladder contraction. For example, contraction of the detrusor muscle may be sensed and interpreted as a precursor to an imminent voiding event if the level of contraction exceeds a predetermined threshold level. In this case, stimulator 18 may quickly increase the stimulation level to a level intended to stop or prevent an involuntary voiding event. The stimulation level may be increased in discrete steps or in proportion to the level of the contraction. The stimulation level may be decreased gradually if contractions subside.

To sense both gradual deformation and instantaneous contractions as bladder activity or condition signals, processing circuitry within sensor 16, or stimulator 18 or programmer 22, may apply two different processing schemes. For gradual deformation, indicative of a fill stage, the present deformation level may be compared to a threshold level, on an absolute basis. For rapid contraction, however, the applicable threshold level may be dynamic. In particular, the threshold level for contractions may be adjusted as the gradual deformation level increases or decreases. In this manner, a contraction may be detected as a rapid change in deformation, relative to the present deformation level.

If the deformation level has gradually increased from a baseline to level X, then level X can be correlated with a fill stage. A rapid contraction then can be sensed by determining whether the deformation level rapidly increases to X+A, where A represents the amount of deformation associated with a detrusor muscle contraction. Hence, gradual deformation may be correlated with a fill stage on an absolute basis, whereas contraction may be determined as the delta between the steady state deformation level and an instantaneous change in the deformation level. Suitable processing electronics, including appropriate comparator, filter, and sample and hold circuitry, may be provided in sensor 16 to sense both fill stage and instantaneous muscle contractions.

As an alternative, two different sensing elements may be used to sense bladder fill stage and contraction. In this case, the contraction threshold level need not be dynamic, and may be configured to respond only to higher frequency changes in deformation. As a further alternative, in some embodiments, sensing of contractions may be correlated with a particular fill stage. If it is assumed that detrusor muscle contractions will begin to occur at a particular fill stage, for example, then sensing of contractile activity may be interpreted as a fill stage of bladder 14. Accordingly, delivery of stimulation may be adjusted in response to a fill stage as determined by absolute deformation level, or as a fill stage determined by the onset of bladder contractions. In either case, stimulator 18 is able to react to bladder condition or activity signals and thereby adjust stimulation levels to avoid involuntary voiding.

Sensor housing 24 may be made from a biocompatible material such as titanium, stainless steel or nitinol, or a polymeric material such as silicone or polyurethane. Another material for fabrication of sensor housing 24 is a two-part epoxy. An example of a suitable epoxy is a two-part medical implant epoxy manufactured by Epoxy Technology, Inc., mixed in a ratio of 10 grams of resin to one gram of activator. In general, sensor housing 24 contains no external openings, with the exception of the opening containing sensing element 30, thereby protecting power source 28 and circuit board 26 from the environment within bladder 14. The opening in sensor housing 24 that receives sensing element 30 is sealed to prevent exposure of interior components.

In some embodiments, sensor housing 24 may have a capsule-like shape with a length in a range of approximately 2 to 15 mm, a width in a range of approximately 2 to 10 mm, and a height in a range of approximately 2 to 10 mm. The capsule-like shape may produce a circular cross-section, in which case sensor housing 24 may have a diameter of approximately 3 to 10 mm, rather than width and height dimensions. Vacuum cavity 34 may be sized to capture a volume of bladder wall tissue on the order of approximately 1 to 5 mm³.

Inward deflection of sensing element 30 may signal the expansion of bladder 14. This expansion may be due to the gradual addition of urine in the bladder or a contraction of muscle in bladder wall 38. During expansion of bladder 14, stimulator 18 may provide electrical stimulation to enhance pelvic floor tone or urinary sphincter function, for example, to keep urine within the bladder. Once sensing element 30 indicates a sufficiently large expansion, electronics on circuit board 26 generate bladder information based on the expansion. Bladder sensor 16 may communicate the information directly to external programmer 22 or stimulator 18 by wireless telemetry. In other embodiments, sensor 16 may be coupled to implantable stimulator 18 by a wired connection.

In response to a signal from sensor 16, stimulator 18 may activate stimulation or increase stimulation intensity to maintain or increase pelvic floor tone or urinary sphincter pressure, and thereby prevent an involuntary voiding event. Stimulation intensity may be increased or decreased by adjusting one or more stimulation parameters such as amplitude (current or voltage), pulse width, pulse rate, electrode combination or polarity. External programmer 22 may signal patient 12 that bladder 14 should be voided. Once stimulator 18 receives confirmation from patient 12 to void bladder 14, e.g., by depression of a button on programmer 22, the stimulator may temporarily cease stimulation, reduce stimulation intensity, or maintain the present level of intensity to allow urine to exit bladder 14.

Alternatively, bladder sensor 16 may automatically monitor the contraction of bladder 14 to signal the end of voiding and the restart of stimulation. As a further alternative, when a patient indicates an intent to void, e.g., by entry of a voiding command into programmer 22, stimulator 18 or programmer 22 may apply a blanking interval to bladder sensor 16. During the blanking interval, bladder sensor 16 does not generate bladder condition signals, or stimulator 18 or programmer 22 ignores bladder condition signals. Consequently, stimulation is not inadvertently adjusted during the blanking interval due to intentional bladder contractions initiated by the patient for voiding. In either case, bladder sensor 16, stimulator 18 and external programmer 22 may serve to prevent involuntary leakage and provide the patient with sufficient time to arrive at a restroom for voluntary voiding, either directly or by catheterization.

For spinal cord injury patients who cannot perceive a sensation of bladder fullness, other embodiments may involve bladder sensor 16 being utilized without implantable stimulator 18. Bladder sensor 16 may be provided to communicate the current status of bladder 14 to external programmer 22 which signals patient 12 as to the status of the bladder. External programmer 22 may contain an LCD, LED lights, other display, audio feedback or tactile feedback. The feedback may inform patient 12 as to how full bladder is or if it is time to urinate to avoid an incontinence event or avoid a dangerously high bladder pressure that could result in kidney problems. Moreover, patient 12 may utilize system 10 for planning the ingestion of solid or liquid food. For example, if bladder 14 is becoming full and bladder voiding is not possible shortly, patient 12 may stop any drinking or eating activities to help avoid an incontinence event.

Adjustment of stimulation parameters by stimulator 18 may be responsive to bladder information transmitted by implantable bladder sensor 16. For example, external programmer 22 or implantable stimulator 18 may adjust stimulation parameters, such as amplitude, pulse width, and pulse rate, or electrode combination and polairty, based on bladder information received from implantable sensor 16. Stimulator 18 may adjust the stimulation parameters autonomously, or under control in response to command signals transmitted by external programmer 22. In either case, implantable stimulator 18 adjusts stimulation to either increase or reduce pelvic floor muscle tone or urinary sphincter contraction based on the actual bladder expansion or contraction. Bladder sensor 16 may transmit bladder information substantially continuously or periodically, e.g., every few seconds or minutes. In some embodiments, bladder sensor 16 may transmit bladder information when there is an abrupt change sensed by sensing element 30, e.g., a deformational change that exceeds a predetermined threshold, indicating a contraction.

Stimulator 18 may respond to abrupt indications of bladder contraction. Alternatively, or additionally, stimulator 18 may respond to more gradually changing, periodic signals indicating that bladder volume has exceeded a threshold. In each case, stimulator 18 may adjust electrical stimulation parameters, such as amplitude, pulse width or pulse rate, to prevent involuntary voiding. In addition to parameter adjustments, adjustment may involve on and off cycling of the stimulation in response to sensed bladder size indicative of a particular bladder fill stage. For example, stimulation may be turned off until the sensed bladder size or volume exceeds a threshold indicative of a particular fill stage of the bladder, at which time stimulation is turned on. At the same time, stimulation parameters may be further adjusted as the sensed bladder size continues to increase. If an abrupt contraction is sensed by sensor 16, stimulator 18 may respond quickly to increase stimulation intensity and thereby prevent involuntary voiding.

External programmer 22 may be a small, battery-powered, portable device that accompanies patient 12 throughout a daily routine. Programmer 22 may have a simple user interface, such as a button or keypad, and a display or lights. Patient 12 may voluntarily initiate a voiding event, i.e., a voluntary voiding of bladder 14, via the user interface. In this case, programmer 22 may transmit a command signal to stimulator 18 to temporarily suspend stimulation, and thereby permit voluntary voiding. In some embodiments, the length of time for a voiding event may be determined by pressing and holding down a button for the duration of a voiding event, pressing a button a first time to initiate voiding and a second time when voiding is complete, or by a predetermined length of time permitted by programmer 22 or implantable stimulator 18. In each case, programmer 22 causes implantable stimulator 18 to temporarily suspend stimulation so that voluntary voiding is possible.

Implantable stimulator 18 may be surgically implanted at a site in patient 12 near the pelvis. The implantation site may be a subcutaneous location in the side of the lower abdomen or the side of the lower back or upper buttocks. One or more electrical stimulation leads 20 are connected to implantable stimulator 18 and surgically or percutaneously tunneled to place one or more electrodes carried by a distal end of the lead at a desired nerve site, such as a sacral nerve site within the sacrum. Stimulator 18 has a biocompatible housing, which may be formed from titanium, stainless steel or the like. Stimulator 18 may be configured to deliver electrical stimulation pulses with a range of electrical parameter values, such as amplitude, pulse width and pulse rate, selected to prevent involuntary leakage of urine from bladder 14.

Attaching implantable bladder sensor 16 to the bladder wall 38 of bladder 14 may be accomplished in a variety of ways, but preferably is completed in a manner that will not excessively injure bladder 14 or otherwise cause excessive trauma during implantation. Preferably, attachment should cause limited inflammation and substantially no adverse physiological modification, such as tissue infection or a loss in structural integrity of bladder 14. However, it is desirable that implantable bladder sensor 16 also be attached securely to the attachment site in order to provide an extended period of measurement without prematurely loosening or detaching from the intended location.

As an example, sensor housing 24 may contain a vacuum cavity 34 that permits a vacuum to be drawn by a vacuum channel 32. The vacuum is created by a deployment device having a vacuum line in communication with vacuum channel 32. The vacuum draws tissue 40 from bladder wall 38 into vacuum cavity 34. Once tissue 40 of bladder wall 38 is captured within vacuum cavity 34, a fastening pin 36 is driven into the captured tissue to attach sensor housing 24 within bladder 14. Fastening pin 36 may be made from, for example, stainless steel, titanium, nitinol, or a high density polymer.

The shaft of pin 36 may be smooth or rough, and the tip may have a sharp point to allow for easy penetration into tissue. Fastening pin 36 may be driven into housing 24 and tissue 40 of bladder wall 38 under pressure, or upon actuation by a push rod, administered by a deployment device. In another embodiment, implantable bladder sensor 16 may be attached without the use of a penetrating rod but with a spring-loaded clip to pinch trapped bladder wall 38 within cavity 34. A variety of other attachment mechanisms, such as pins, clips, barbs, sutures, helical screws, surgical adhesives, and the like may be used to attach sensor housing 24 to bladder wall 38 of bladder 14.

In the example of FIGS. 1 and 2, sensor housing 24 of implantable bladder sensor 16 is attached to the exterior bladder wall 38 of bladder 14 near the side of the bladder. However, the attachment site for sensor housing 24 could be at any position on bladder wall 38 that does not interfere with bladder function or other organ function. For example, sensor housing 24 may be placed in the top of the bladder or near the urethra. In some patients, the most desirable position may coincide with the least invasive implantation surgery. Bladder sensor 16 may be surgically implanted using open surgery or laparoscopic techniques.

FIG. 3 is an enlarged, bottom view of the implantable bladder sensor of FIG. 2. Sensor housing 24 includes sensing element 30 and vacuum cavity 34, which come into contact with bladder wall 38. While sensing element 30 is rectangular and large with respect to sensor housing 24 to contact a large surface area of bladder wall 38, some embodiments may include two or more sensing elements, such as strain gauges of similar or different shapes. For example, housing 24 may include a sensing element on each end of housing 24 separated by vacuum cavity 34.

Vacuum cavity 34 holds a portion of tissue from bladder wall 38 in order to keep sensing element 30 in contact with the exterior surface of bladder 14. In some embodiments, sensor housing 24 may contain more than one vacuum cavity to attach to multiple points along bladder wall 38. For example, one vacuum cavity on each end of housing 24 may provide secure contact between sensing element 30 and bladder wall 38. In other embodiments, housing 24 may be formed into a different shape than a rectangle. For example, housing 24 may comprise a circular shape or concave shape to better fit the curvature of bladder 14.

FIG. 4 is a cross-sectional side view of a distal portion of a deployment device 41 used for deployment and fixation of an implantable bladder sensor 16. In the example of FIG. 4, deployment device 41 includes a distal head 42. Distal head 41 may be mounted on an elongated sheath 43 (partially shown in FIG. 4) configured for laparoscopic introduction into patient 12 through a trocar. Deployment device 41 may be used with other laparoscopic components, such as a gas distension tube for inflating the pelvic cavity to facilitate access to bladder 14, and a visualization scope for viewing the implantation site. In some embodiments, visualization components may be integrated with deployment device 41.

As shown in FIG. 4, distal head 42 receives a vacuum line 44 and a positive pressure line 46 via elongated sheath 43. Vacuum line 44 is coupled to a vacuum outside of patient 12 via a tube or lumen extending along the length of deployment device 41. Similarly, positive pressure line 46 is coupled to a positive pressure source (not shown) via a tube or lumen extending along the length of deployment device 41. Vacuum line 44 is in fluid communication with vacuum channel 32 and vacuum cavity 34, and permits the physician to draw a vacuum and thereby capture tissue 40 of bladder wall 38 within the vacuum cavity. Positive pressure line 46 permits the physician to apply a pulse of high pressure fluid, such as a liquid or a gas, to drive fixation pin 36 into sensor housing 24 and through tissue 40 of bladder wall 38. Pin 36 thereby fixes sensor housing 24 to external bladder wall 38. In some embodiments, a membrane mounted over an opening of positive pressure line 46 may be punctured by pin 36.

Once fixation pin 36 attaches sensor 16 to bladder 14, vacuum line 44 is no longer needed. However, in some embodiments, vacuum line 44 may be used to detach pressure sensor 16 from distal head 42 of deployment device 41. By terminating vacuum pressure, or briefly applying positive pressure through vacuum line 44, for example, head 42 may separate from sensor 16 due to the force of the air pressure. In this manner, vacuum line 44 may aid in detachment of sensor 16 prior to removal of deployment device 41.

As described previously in FIG. 2, fixation pin 36 punctures bladder wall 38 for fixation of sensor 16. While the force of this fixation may vary with patient 12, deployment device 41 provides adequate force for delivery of pin 36. In an exemplary embodiment, positive pressure line 46 is completely sealed and filled with a biocompatible fluid (such as water, saline solution or air). Sealing the end of positive pressure line 44 is fixation pin 36 or a head on fixation pin 36.

Fixation pin 36 is generally able to move within positive pressure line 46 much like a piston. Force to push fixation pin 36 through tissue 40 of bladder wall 38 captured in vacuum cavity 34 is created by application of a pulse of increased fluid pressure within positive pressure line 46. For example, the physician may control a positive pressure source via control handle attached to deployment device 41. This simple delivery method may provide high levels of force, allow multiple curves and bends in deployment device 41, and enable a positive pressure line 46 of many shapes and sizes.

In an alternative embodiment, a flexible, but generally incompressible, wire may be placed within positive pressure line 46 and used as a push rod to force fixation pin 36 through the captured tissue 40 of bladder wall 38. This wire presents compressive force from the control handle of deployment device 41 directly to fixation nail 36. This method may eliminate any safety risk of pressurized fluids entering patient 12 or, in some embodiments, permit retraction of pin 36 after an unsuccessful fixation attempt. If attached, the flexible wire may be attached to pin 36 and pulled back to remove the pin from tissue 40. The flexible wire may be sheared from fixation pin 36 for detachment purposes as distal head 42 releases sensor 16. This detachment may be facilitated by a shearing element or low shear stress of the wire.

In FIG. 4, deployment device 41 illustrates the attachment of vacuum line 44 and positive pressure line 46 to one end of sensor 16. In some embodiments, deployment device 41 may attach vacuum line 44 and positive pressure line 46 to their respective channels opening on the top of sensor housing 42 instead of the side of sensor housing 42. This change in location may facilitate attachment of sensor 16 from a variety of locations or on certain locations on the outside of bladder 14.

Deployment device 41 is introduced to patient 12 by a small incision in the abdomen of the patient. A surgeon may guide distal head 42 through the abdominal space to the exterior of bladder 14. Once at bladder 14, the surgeon locates the desired spot for attaching sensor 16. Sensor 16 is then pressed up against bladder wall 38 and the vacuum is initiated to bring tissue 40 into vacuum cavity 34 before fixation pin 36 is driven through tissue 40. Deployment device releases sensor 16 and is removed from patient 12.

In other embodiments, sensor 16 may be attached to bladder 14 through open abdominal surgery to precisely locate the attachment point on bladder 14. In this type of procedure, deployment device 41 may or may not be used to attach sensor 16 to bladder wall 38. In some embodiments, deployment device 41 may include a small endoscopic camera in the distal head 42. The camera may enable the physician to better guide deployment device 41 through a small opening in patient 12 to a desired attachment location on the external surface of bladder 14 in less time with more accuracy, as is common in endoscopic surgery. Images may be displayed using video fed to a display monitor.

Distal head 42 may be disposable. Disposable devices that come into contact with patient 12 tissues and fluids greatly decrease the possibility of infection in implantable devices. In other embodiments, the entire deployment device 41 may be manufactured from robust materials intended for multiple uses. The device would then need to be sterilizable between uses. In still a further embodiment, the features of distal head 42 may be incorporated into bladder sensor 16. In this configuration, bladder sensor 16 may be larger in size but would include the necessary elements for attachment within the device. After attachment, the entire sensor would detach from the handle of deployment device 41, reducing the difficulty of removing the entire deployment device 41, including distal head 42.

After the useful life of implantable bladder sensor 16 is complete or it is no longer needed within patient 12, it can be removed from patient 12 in some manner. Alternatively, sensor 16 may simply remain in place. As an example, deployment device 41 may be reinserted into patient 12, navigated to bladder 14, and reattached to bladder sensor 16. Deployment device 41 may then be withdrawn from bladder 14, explanting sensor 16 from patient 18. Alternatively, a surgeon may perform open abdominal surgery to remove the implanted bladder sensor 16 and stimulator 18.

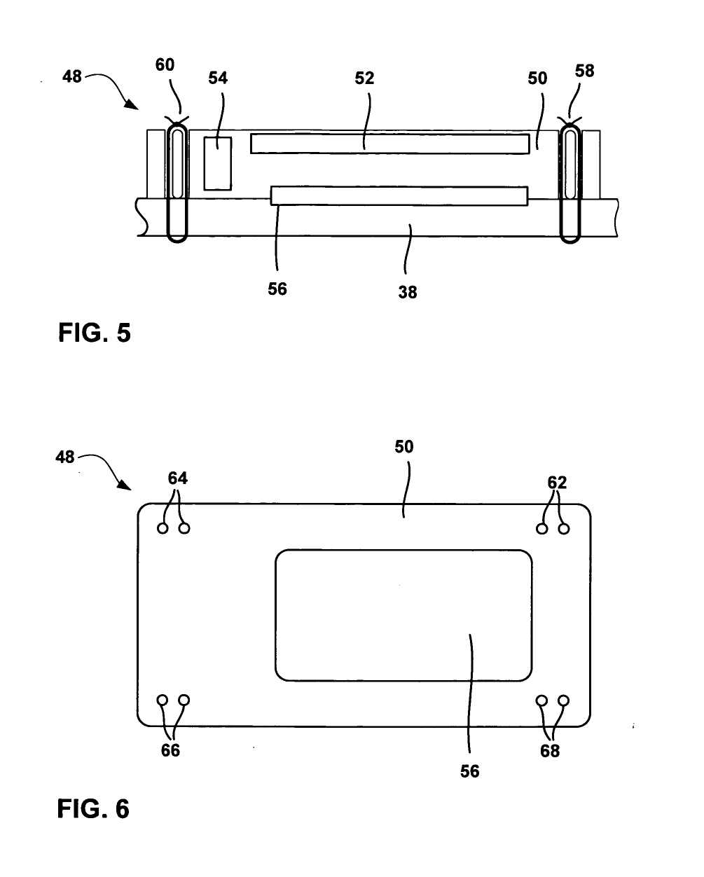

FIG. 5 is an enlarged schematic diagram illustrating an implantable bladder sensor sutured to the outside of the bladder of a patient. Sensor housing 50 is attached to bladder wall 38 and includes circuit board 52, power source 54, and sensing element 56. Sutures 58 and 60 are used to attach bladder sensor 48 to bladder wall 38. Although only two sets of sutures can be shown in FIG. 5, sensor 48 may include four or more sets, one at each corner of the rectangular shaped sensor.

Circuit board 52, power source 54 and sensing element 56 may all be similar to circuit board 26, power source 28 and strain gauge 30 of FIG. 2. In addition, sensor housing 50 may be functionally similar to sensor housing 24 of FIG. 2. Differences between these components of each embodiment may relate to only the size or shape of each component. As in some embodiments of sensing element 30, sensing element 56 may include a strain gauge sensor that senses a change in deformation of bladder wall 38 as bladder 14 expands and contracts. Sensing element 56 sends the bladder information to circuit board 52. Circuit board 52 wirelessly communicates the bladder information to stimulator 18, external programmer 22, or both. Circuit board 52 also may control the operation of sensor 48.

Bladder sensor 48 may be implanted through laparoscopic techniques, similar to bladder sensor 16. For example, a surgeon may make a few small incisions in the abdomen of patient 12 and guide bladder sensor 48 to bladder 14 with the aid of a small camera. Once sensor 48 is placed on the external surface bladder wall 38, the surgeon uses sutures to tie sensor 48 to bladder wall 38, which is illustrated by sutures 58 and 60 in FIG. 5. The sutures may or may not penetrate through bladder wall 38, and no urine will escape bladder 14 in either case.

In other embodiments, bladder sensor 48 may be implanted through more invasive procedures, such as open abdominal surgery which exposes bladder 14. In some embodiments, metal or plastic staples may be used to fix sensor 16 to bladder wall 38 instead of nylon sutures. In some embodiments, multiple sensors 48 may be placed around bladder 14 to generate an average expansion or contraction of the entire bladder.

Once attached to bladder wall 38, sensing element 56 may be securely forced against bladder wall 38. As bladder 14 expands and contracts, sensing element 56 may sense the changed pressure by bladder wall 38 and indicate a change in size of the bladder. Similar to sensing element 30 of FIG. 2, many other types of sensing components may be used to sense a change in deformation of bladder 14. However, a strain gauge is described herein for purposes of illustration.

FIG. 6 is an enlarged, bottom view of the implantable bladder sensor of FIG. 5. Bladder sensor 48 includes sensor housing 50 and sensing element 56. Fixation holes 62, 64, 66 and 68 are voids in housing 50 and allow suture to be passed through housing 50 in order for sensor 48 to be attached to bladder wall 38. Sensing element 56 may occupy a majority of the surface area of bladder sensor 48 that contacts bladder wall 38. While sensing element 56 is rectangular in shape, the strain gauge may be formed of any symmetric or asymmetrical shape. In the example of FIGS. 5 and 6, sensor 48 may have a patch-like shape, and may have a length of approximately 2 to 15 mm, a width of approximately 2 to 10 mm, and a thickness of approximately 2 to 10 mm.

Fixation holes 62, 64, 66 and 68 each contain a pair of passages through housing 50. Each pair of passages is located near a corner of housing 50. A surgeon may pass a suture through these holes to attach housing 50 to bladder 14 in a desired location of bladder wall 38. While fixation holes 62, 64, 66 and 68 each contain two holes, other embodiments may include more or less holes in housing 50. For example, each corner of housing 50 may only contain one hole. Suture would then pass through the hole and around the outside of housing 50. As a further example, each corner may contain three holes for further securing housing 50 to bladder wall 38.

Other fixation methods to secure bladder sensor 48 to bladder wall 38 may include other structures different than sutures. For example, each corner of housing 50 may contain a barbed needle or helical screw that ejects from housing 50 into bladder wall 38. The barbed needles may secure sensor 48 to bladder 14 without lengthy attachment procedures. Also, surgical adhesives may be used as an alternative, or in addition to, mechanical fasteners such as sutures, needles or screws.

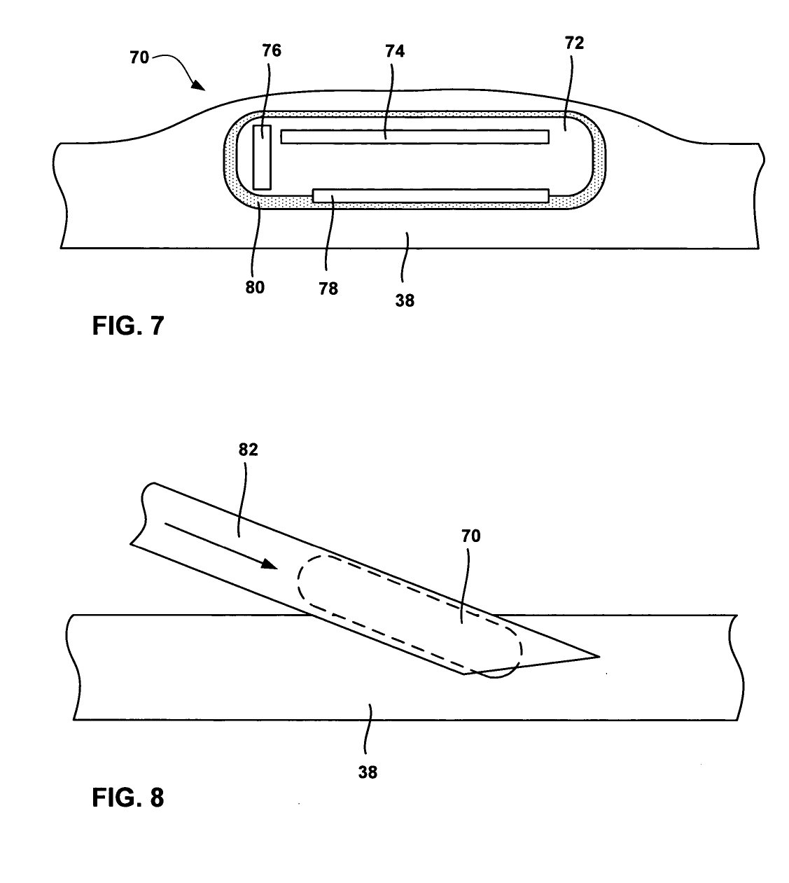

FIG. 7 is a cross-sectional side view of an implantable bladder sensor placed within the bladder wall 38 of a patient 12. Sensor housing 72 of bladder sensor 70 is embedded in bladder wall 38 and includes circuit board 74, power source 76, and sensing element 78. Sensor housing 72 is in the shape of a rounded capsule and includes a smooth surface. The only structure extending from housing 72 is a sensing element 78, such as a strain gauge, which slightly protrudes from the housing to sense deformation changes in bladder wall 38. Sensor 70 rests in wall cavity 80 formed within bladder wall 38. Sensor 70 may have a capsule-like shape, and may have a length of approximately 2 to 10 mm, a width of approximately 2 to 5 mm, and a thickness of approximately 1 to 5 mm. The capsule-like shape may produce a circular cross-section, in which case sensor 70 may have a diameter of approximately 1 to 5 mm, rather than width and height dimensions.

Circuit board 74, power source 76 and sensing element 78 may be similar to respective circuit board 26, power source 28 and sensing element 30 of FIG. 2. In addition, sensor housing 72 may be functionally similar to sensor housing 24 of FIG. 2. Differences between these components of each embodiment may relate to the size or shape of each component. Therefore, sensing element 78 senses a change in deformation of bladder wall 38 as bladder 14 expands and contracts. Processing electronics on circuit board 74 detect these changes sensed by sensing element 78. Circuit board 74 communicates the bladder information to stimulator 18, external programmer 22, or both, e.g., by wireless telemetry. Circuit board 74 also controls the operation of sensor 70.

Implanting bladder sensor 70 within bladder wall 38 may be a simple method for securing the sensor sensing element 78. As bladder 14 expands and contracts, sensing element 78 senses the changed pressure of bladder wall 38 and indicates a change in size of the bladder or an abrupt contraction. For example, a higher force in bladder wall 38 may indicate an expanding bladder 14 or a contraction. Although sensing element 78 may be a strain gauge, many other types of sensing components may be used to sense a change in deformation of bladder 14.

FIG. 8 is a schematic diagram illustrating the endoscopic deployment of the implantable bladder sensor of FIG. 7. Bladder sensor 70 may be implanted through endoscopic, laparoscopic, or similar minimally invasive techniques. A surgeon makes a few small incisions in the abdomen of patient 12 and guides bladder sensor 70 within needle 82 to bladder 14 with the aid of a small camera. Needle 82 may be constructed of a metal alloy and comprise a hollow cylinder and a pointed distal end for puncturing bladder wall 38. Needle 82 includes bladder sensor 70 and a fluid to force the sensor out of the needle. An exemplary fluid may be saline or other biocompatible fluid. In other embodiments, needle 82 may comprise a catheter or other hollow delivery vehicle.

Once needle 82 in positioned at the appropriate location of bladder 14, the surgeon may force sensor 70 into place. Removing needle 82 from bladder wall 38 allows the external tissue of bladder wall 38 to close and surround sensor 70. In some embodiments, the surgeon may suture the insertion hole of bladder wall 38 to promote tissue healing. The suture may comprise resorbable or non-resorbable suture or staples. When implanting sensor 70, the inner surface of bladder wall 38 should not be breached in order to prevent patient 12 from developing infection or other health problems.

In other embodiments, bladder sensor 70 may be implanted through more invasive procedures, such as open abdominal surgery which exposes bladder 14. In some embodiments, multiple sensors 70 may be placed around bladder 14 to generate an average expansion or contraction of the entire bladder.

Bladder sensor 70 has a biocompatible housing, which may be formed from titanium, stainless steel or other materials. In some embodiments, bladder sensor 70 may carry one or more expandable elements that help to anchor the sensor within the bladder wall. The expandable elements may be constructed from a hydrogel material. During implantation, the expandable elements are in a dehydrated state, in which the expandable elements are smaller. But when implanted in the body of a patient, the expandable elements absorb water from the body tissues and assume a hydrated state. In the hydrated state, the expandable elements have a larger perimeter. Expansion of the expandable elements resists migration of the sensor 70 within bladder wall 38.

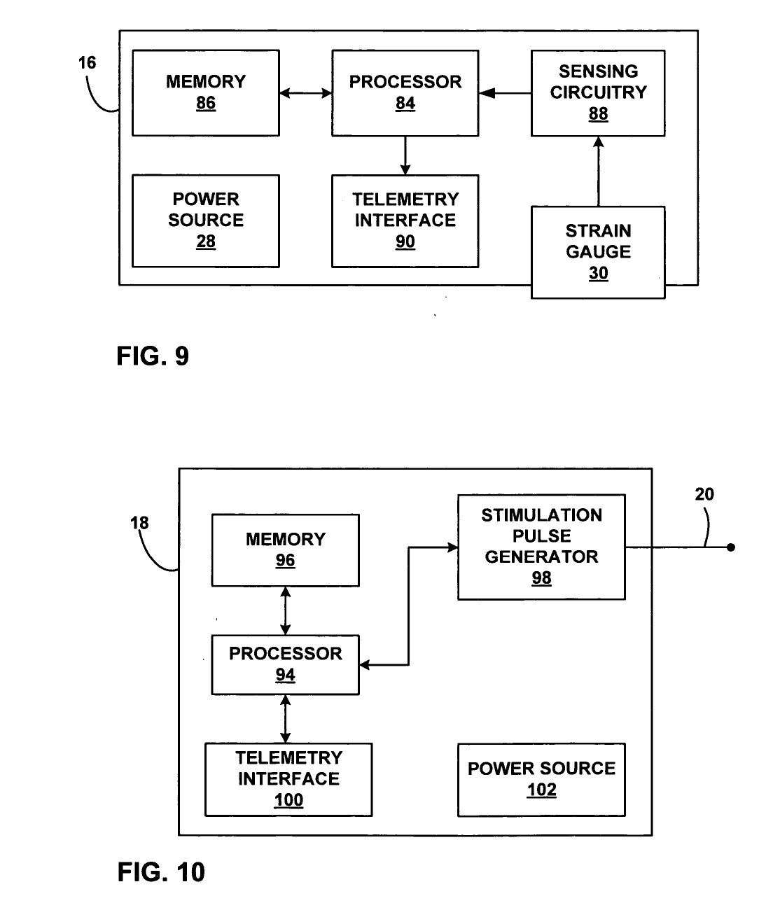

FIG. 9 is a functional block diagram illustrating various components of an exemplary implantable bladder sensor 16 (FIG. 2), 48 (FIG. 5) or 70 (FIG. 7). In the example of FIG. 9, implantable bladder sensor 16 includes a processor 84, memory 86, sensing circuitry 88, telemetry interface 90, power source 28 and strain gauge sensing element 30. Sensing circuitry 88 may be carried on a circuit board, along with processor 84, memory 86 and telemetry interface 90. Strain gauge 30 transforms mechanical deformation from bladder 14 into electrical signals representative of bladder size or contraction. The electrical signals may be amplified, filtered, and otherwise processed as appropriate by sensing circuitry 88 within sensor 16. In some embodiments, the signals may be converted to digital values and processed by processor 84 before being saved to memory 86 or sent to implantable stimulator 18 as pressure information via telemetry interface 90.

Memory 86 stores instructions for execution by processor 84 and bladder information generated by sensing circuitry 88. Bladder data may then be sent to implantable stimulator 18 or external programmer 22 for long-term storage and retrieval by a user. Memory 86 may include separate memories for storing instructions and bladder information. In addition, processor 84 and memory 86 may implement loop recorder functionality in which processor 84 overwrites the oldest contents within the memory with new data as storage limits are met, thereby conserving data storage resources within pressure sensor 16.

Processor 84 controls telemetry interface 90 to send bladder information to implantable stimulator 18 or programmer 22 on a continuous basis, at periodic intervals, or upon request from the implantable stimulator or programmer. Wireless telemetry may be accomplished by radio frequency (RF) communication or proximal inductive interaction of bladder sensor 16 with programmer 22.

Power source 28 delivers operating power to the components of implantable bladder sensor 16. As mentioned previously, power source 28 may include a small rechargeable or non-rechargeable battery and a power generation circuit to produce the operating power. Recharging may be accomplished through proximal inductive interaction between an external charger and an inductive charging coil within sensor 16. In some embodiments, power requirements may be small enough to allow sensor 16 to utilize patient motion and implement a kinetic energy-scavenging device to trickle charge a rechargeable battery. In other embodiments, traditional batteries may be used for a limited period of time. As a further alternative, an external inductive power supply could transcutaneously power sensor 16 whenever pressure measurements are needed or desired.

FIG. 10 is a functional block diagram illustrating various components of an implantable stimulator 18 which communicates with an implantable bladder sensor 16 via wireless telemetry. In the example of FIG. 10, stimulator 18 includes a processor 94, memory 96, stimulation pulse generator 98, telemetry interface 100, and power source 102. Memory 96 may store instructions for execution by processor 94, stimulation therapy data, and bladder information received from bladder sensor 16 via telemetry interface. Bladder information is received from bladder sensor 16 and may be recorded for long-term storage and retrieval by a user, or adjustment of stimulation parameters, such as amplitude, pulse width or pulse rate. Memory 96 may include separate memories for storing instructions, stimulation parameter sets, and bladder information.

Processor 94 controls stimulation pulse generator 98 to deliver electrical stimulation therapy via one or more leads 20. Processor 94 controls telemetry interface 100 to send and receive information. An exemplary range of neurostimulation stimulation pulse parameters likely to be effective in treating incontinence, e.g., when applied to the sacral or pudendal nerves, are as follows:

1. Frequency: between approximately 0.5 Hz and 500 Hz, more preferably between approximately 5 Hz and 250 Hz, and still more preferably between approximately 10 Hz and 50 Hz.

2. Amplitude: between approximately 0.1 volts and 50 volts, more preferably between approximately 0.5 volts and 20 volts, and still more preferably between approximately 1 volt and 10 volts.

3. Pulse Width: between about 10 microseconds and 5000 microseconds, more preferably between approximately 100 microseconds and 1000 microseconds, and still more preferably between approximately 180 microseconds and 450 microseconds.

Based on bladder information received from sensor 16, processor 94 interprets the information and determines whether any therapy parameter adjustments should be made. For example, processor 94 may compare the bladder expansion or contraction to one or more thresholds, and then take action to adjust stimulation parameters based on the bladder information. Information may be received from sensor 16 on a continuous basis, at periodic intervals, or upon request from stimulator 18 or external programmer 22. Alternatively, or additionally, bladder sensor 16 may transmit bladder information when there is an abrupt change in the bladder dimensions, e.g., indicating contraction at the onset of involuntary leakage.

Processor 94 modifies parameter values stored in memory 96 in response to bladder information from sensor 16, either independently or in response to programming changes from external programmer 22. Stimulation pulse generator 98 provides electrical stimulation according to the stored parameter values via one or more leads 20 implanted proximate to a nerve, such as a sacral nerve. Processor 94 determines any parameter adjustments based on the bladder information obtained form sensor 16, and loads the adjustments into memory 96 for use in delivery of stimulation.

As an example, if the bladder information indicates a contraction of bladder 14 without the approval of patient 12, processor 94 may increase the amplitude, pulse width or pulse rate, or change electrode combination or polarity, of the electrical stimulation applied by stimulation pulse generator 98 to increase stimulation intensity, and thereby increase sphincter closing pressure or pelvic floor tone. If bladder size stays constant, processor 94 may implement a cycle of downward adjustments in stimulation intensity until bladder contraction is evident, and then incrementally increase the stimulation upward until expansion begins. In this way, processor 94 converges toward an optimum level of stimulation for purposes of patient comfort and power efficiency. Although processor 94 is described as adjusting stimulation parameters, adjustments alternatively may be generated by programmer 22 and transmitted to stimulator 18 as parameter or program changes.

Bladder size or mechanical measurements may change due to a variety of factors, such as an activity type, activity level or posture of the patient 12. Hence, for a given set of stimulation parameters, the efficacy of stimulation may vary in terms of rate of bladder expansion or contraction, due to changes in the physiological condition of the patient. For this reason, the continuous or periodic availability of bladder information from implantable sensor 16 is highly desirable.

With this bladder information, stimulator 18 is able to respond to changes in bladder size with dynamic adjustments in the stimulation parameters delivered to patient 12. In particular, processor 94 is able to adjust parameters in order to improve pelvic floor tone or cause constriction of the urinary sphincter and thereby avoid involuntary leakage. In some cases, the adjustment may be nearly instantaneous, yet prevent leakage. As an example, if patient 12 laughs, coughs, or bends over, the resulting force on bladder 14 could overcome the closing pressure of the urinary sphincter. If bladder sensor 16 indicates an abrupt change in bladder contraction, however, stimulator 14 can quickly respond by more vigorously stimulating the sacral nerves to increase sphincter closing pressure.

In general, if bladder 14 is contracting for an unknown reason, processor 94 may dynamically increase the level of therapy to be delivered to prevent or stop the voiding of bladder 14. Conversely, if bladder 14 is expanding consistently, processor 94 may incrementally reduce stimulation, e.g., to conserve power resources, until the bladder reaches a fill stage that correlates with the need to void and, thus, a possible incontinence event. Increases or reductions in the level of therapy may include upward or downward adjustments in amplitude (current or voltage), pulse width, or pulse rate of stimulation pulses delivered to the patient.

As in the case of sensor 16, wireless telemetry in stimulator 18 may be accomplished by radio frequency (RF) communication or proximal inductive interaction of stimulator 18 with implantable bladder sensor 16 or external programmer 22. Accordingly, telemetry interface 100 may be similar to telemetry interface 90. Also, power source 102 of stimulator 18 may be constructed somewhat similarly to power source 28. For example, power source 102 may be a rechargeable or non-rechargeable battery, or alternatively take the form of a transcutaneous inductive power interface.

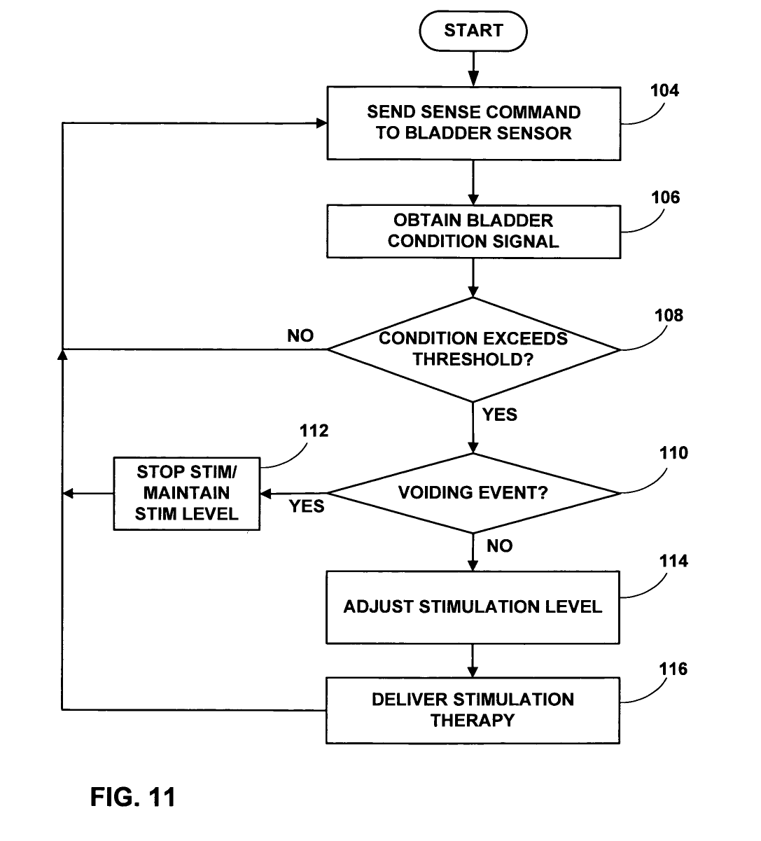

FIG. 11 is a flow chart illustrating a technique for delivery of stimulation therapy based on closed loop feedback from an implantable bladder sensor. In the example of FIG. 11, implantable stimulator 18 requires information from implantable bladder sensor 16 and external programmer 22. The flow of events begins with implantable stimulator 18 communicating with implantable bladder sensor 16 and sending a command to sense the stretch, deformation or contraction of bladder 14 (104). The sending of a sense command may be optional. For example, in other embodiments, bladder sensor 16 may voluntarily sense a bladder condition on a periodic basis and provide a bladder condition signal to stimulator 18 or programmer 22 on a periodic or polled basis.

Bladder sensor 16 subsequently acquires a bladder condition measurement and delivers a bladder activity signal to implantable stimulator 18 (106), e.g., by wireless telemetry. Alternatively, the data may be transmitted from sensor 16 to external programmer 22. Upon receiving the bladder activity signal, implantable stimulator 18 calibrates the data and compares it to a determined threshold (108). If the measured bladder condition does not exceed the applicable threshold value, the loop begins again. If the measured condition exceeds the threshold, indicating an advanced fill stage or a contraction, the flow continues to the next step of stimulation. In some embodiments, threshold comparisons may be provided for both fill stage and contraction level. If either fill stage or contraction level exceeds an applicable threshold, the stimulation level may be adjusted.

The bladder condition may be a signal indicating a level of stretch, deformation or contraction of bladder 14, or an activity state such as fill state or contraction inferred from such levels. If sensor 16 includes a strain gauge sensor, the bladder condition signal may be based on a change in impedance of the strain gauge or a voltage across the strain gauge that varies as a function of the impedance change. The bladder activity signal may be based on a single impedance change sample, or a series of samples over a period of time. Hence, the bladder condition signal may indicate an instantaneous amplitude or a rate of change, i.e., slope, in amplitude over a series of samples, or a combination thereof. Again, as mentioned previously, a single sensor may sense both gradual deformation, for fill stage, and instantaneous deformation changes, for contractions. Alternatively, separate sensors may be used for fill stage and contractions.

As a further alternative, more sophisticated digital signal processing may be used to correlate a series of samples with a waveform or pattern known to be indicative of contraction. The processing of the measurements obtained by a sensing element 30 may be performed by processing electronics and/or software provided onboard with sensor 16, or by processing electronics and/or software provided by stimulator 18 or external programmer 22. Hence, stimulator 18 or external programmer 22 may receive raw sense data from sensor 16, or pre-processed bladder activity signals from sensor 16. In addition, the threshold comparison represented by reference numeral 108 may be performed within stimulator 18 or external programmer 22, or within sensor 16 itself in some embodiments.

Stimulator 18 and programmer 22 may receive sense data from sensor 16 in some embodiments. For example, stimulator 18 may react to instantaneous changes in bladder condition, while programmer 22 may react to changes in bladder condition over a period of time, e.g., trend data. Alternatively, either stimulator 18 or programmer 22 may be configured to react to instantaneous and trending bladder changes.

In some embodiments, implantable stimulator 18 may communicate with external programmer 22 to check if patient 12 has desired to void the contents of bladder 14 (110). If the bladder activity level exceeds an applicable threshold (108), but patient 12 has signaled a voiding event (110), e.g., via external programmer 22, stimulation may be stopped for a brief window of time or maintained at its current stimulation level to enable the patient to urinate (112). The process then begins again and bladder sensing starts once more. In the case in which no voiding event desired, more intense stimulation may be required to counteract bladder contraction. Implantable stimulator 18 performs the necessary tasks to adjust the level of stimulation (114), and thereby increases sphincter closing force or pelvic floor tone. Stimulator 18 concludes the loop by delivering electric stimulation therapy to appropriate nerves (116). After voiding, stimulation may be turned off until bladder 14 reaches a particular fill stage. After stimulation therapy has commenced, the loop begins again to continue appropriate urinary incontinence therapy to patient 12.

In some embodiments, bladder sensor 16 may be used exclusively for monitoring bladder activity without providing feedback for stimulation therapy. In this case, the process represented in FIG. 11 may be much simpler and only include collecting data and sending it to external programmer 22 (104 and 106). Bladder stretch may be measured continuously, intermittently or at the request of stimulator 18 or external programmer 22. These embodiments may be used for disease diagnosis or condition monitoring and may enable patient 12 to avoid frequent clinic visits and uncomfortable procedures. In some embodiments, the bladder measurements may form part of an automated voiding diary that records voluntary voiding events, involuntary voiding events, and bladder activity levels prior to, contemporaneous with, of after such an event.

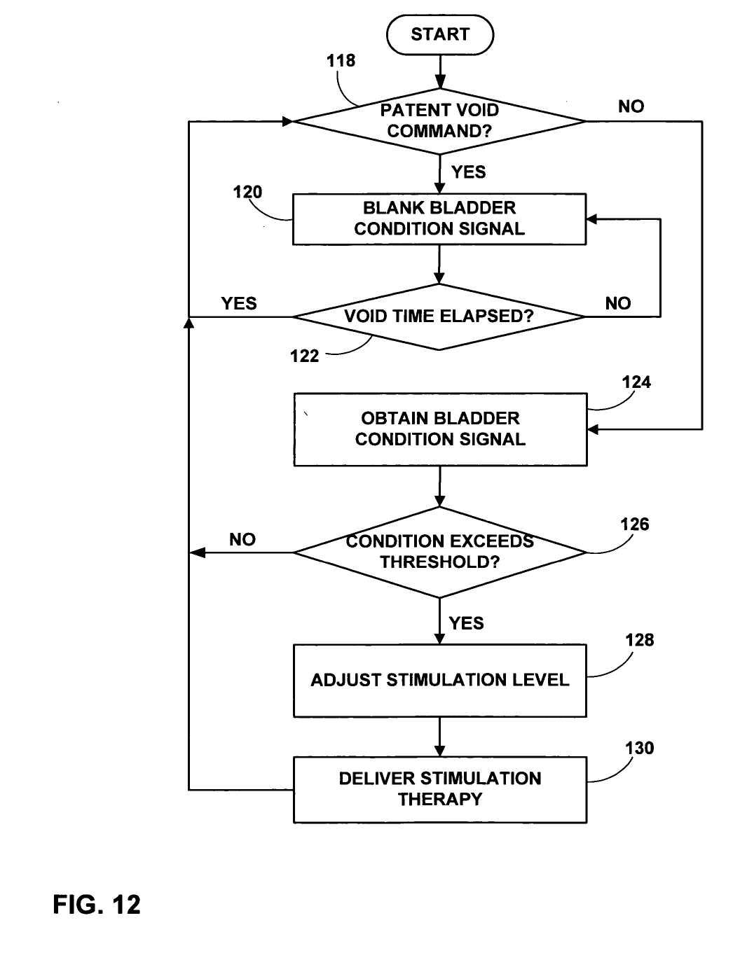

FIG. 12 is a flow chart illustrating an alternative technique for delivery of stimulation therapy based on closed loop feedback from an implantable bladder sensor. In some cases, stimulation may be delivered at a level that prevents unintentional voiding of urine, but permits the patient to intentionally overcome the stimulation to void urine. Accordingly, stimulation does not necessarily need to be stopped for intentional voiding. However, it is desirable that the stimulation level not be increased in response to bladder contraction while the patient is attempting to void urine. For this reason, it may be desirable to apply a blanking interval to sensor 16. The blanking interval is a period during which sensor 16 does not sense bladder activity, or any sensed activity is ignored, so that stimulation is not inadvertently adjusted in response to bladder contraction associated with an intentional voiding event.

As shown in FIG. 12, if a patient void command is received (118), e.g., by user input to an external programmer 22, the programmer 22 applies a blanking interval to the bladder condition signal (120). The blanking interval may be a period during which bladder condition signals produced by sensor 16 are ignored by programmer 22, stimulator 18, or both. Alternatively, during the blanking interval, programmer 22 or stimulator 18 may send a wireless command to actively disable sensor 16 temporarily. Programmer 22 may directly blank sensor 16 or blank the sensor via stimulator 18. The blanking interval may extend for a predetermined period of time known to be sufficient to complete voiding. Once the voiding time has elapsed (122), programmer 22 may again determine whether a patient void command has been entered (118). Another patient void command resets the blanking interval.

If no patient void command has been received (118), sensor 16 obtains the bladder condition signal (124) and provides the signal to programmer 22 or stimulator 18. The bladder condition signal may be provided on a periodic or polled basis. If the condition, such as fill stage or contraction, or either, exceeds an applicable threshold (126), programmer 22 or stimulator 18 adjusts the stimulation level (128), e.g., by adjusting one of more stimulation pulse parameters such as amplitude, pulse width or pulse rate. The level is adjusted to a level sufficient to avoid involuntary voiding, i.e., an incontinence event. Upon delivery of the stimulation therapy with the adjusted stimulation level (130), the process continues. In particular, programmer 22 may react to a patient void command (118) at any time.

Various embodiments of the described invention may include processors that are realized by microprocessors, Application-Specific Integrated Circuits (ASIC), Field-Programmable Gate Arrays (FPGA), or other equivalent integrated or discrete logic circuitry. A processor may also utilize several different types of data storage media to store computer-readable instructions for device operation. These memory and storage media types may include any form of computer-readable media such as magnetic or optical tape or disks, solid state volatile or non-volatile memory, including random access memory (RAM), read only memory (ROM), electronically programmable memory (EPROM or EEPROM), or flash memory. Each storage option may be chosen depending on the embodiment of the invention.

Many embodiments of the invention have been described. Various modifications may be made without departing from the scope of the claims. For example, although the invention has been generally described in conjunction with implantable neurostimulation devices, a bladder sensor may also be used with other implantable medical devices, such as electrical muscle stimulation devices, functional electrical stimulation (FES) devices, and other conditions or disorders. These and other embodiments are within the scope of the following claims.

1. A method comprising:
sensing a bladder condition via a implanted sensor attached to an exterior surface of a bladder; and
generating information based on the sensed bladder condition.

2. The method of claim 1, further comprising transmitting the information to an implanted electrical stimulator that delivers stimulation therapy for urinary incontinence based on the information.

3. The method of claim 1, further comprising transmitting the information to an external programmer that controls an implanted electrical stimulator to deliver stimulation therapy for urinary incontinence based on the information.

4. The method of claim 1, further comprising storing the information within the implanted sensor.

5. The method of claim 1, wherein the implanted sensor includes a strain gauge sensor that senses mechanical deformation of the exterior surface of the bladder.

6. The method of claim 1, further comprising processing the sensed bladder condition to detect expansion of the bladder.

7. The method of claim 1, further comprising processing the sensed bladder condition to detect contraction of the bladder.

8. The method of claim 1, further comprising controlling delivery of electrical stimulation therapy to a patient in response to the information to alleviate urinary incontinence.

9. The method of claim 1, wherein the implanted sensor is fixed to the exterior surface of the bladder by a vacuum cavity that captures a portion of the exterior surface and a fixation element that penetrates the captured portion.

10. The method of claim 1, wherein the implanted sensor is sutured to the exterior surface of the bladder.

11. The method of claim 1, wherein the implanted sensor is at least partially implanted within a wall of the bladder.

12. The method of claim 1, wherein the implanted sensor includes a power supply and wireless telemetry circuit to transmit the information.

13. An implantable electrical stimulation system comprising:
an implantable sensor that senses a condition of a bladder;
a fixation structure that attaches the implantable sensor to an exterior surface of the bladder; and
processing circuitry that generates information based on the sensed bladder condition.

14. The system of claim 13, further comprising an implantable electrical stimulator that delivers stimulation therapy for urinary incontinence based on the information.

15. The system of claim 13, further comprising an external programmer that controls an implantable electrical stimulator to deliver stimulation therapy for urinary incontinence based on the information.

16. The system of claim 15, wherein the external programmer controls the delivery of electrical stimulation therapy to a patient in response to the information and patient input to alleviate urinary incontinence.

17. The system of claim 13, further comprising a memory to store the information within the implantable sensor.

18. The system of claim 13, wherein the implantable sensor includes a strain gauge sensor that senses mechanical deformation of the exterior surface of the bladder.

19. The system of claim 13, wherein the processing circuitry detects expansion of the bladder based upon the sensed bladder condition.

20. The system of claim 13, wherein the processing circuitry detects contraction of the bladder based upon the sensed bladder condition.

21. The system of claim 13, wherein the implantable sensor includes a vacuum cavity that captures a portion of the exterior surface of the bladder and a fixation element that penetrates the captured portion.

22. The system of claim 13, wherein the implantable sensor includes one or more suture elements to suture the sensor to the exterior surface of the bladder.

23. The system of claim 13, wherein the implantable sensor is sized for at least partial implantation within a wall of the bladder.

24. The system of claim 13, wherein the implantable sensor includes a power supply and a wireless telemetry circuit to transmit the information.

25. An implantable medical device comprising:
a sensor housing;
a sensing element, associated with the housing, that senses a condition of a bladder;
a fixation structure that fixes the sensor housing to an exterior surface of the bladder; and
processing circuitry that generates information based upon the sensed bladder condition.

26. The device of claim 25, wherein the sensing element comprises a strain gauge that senses mechanical deformation of the exterior surface of the bladder.

27. The device of claim 25, further comprising a memory to store the information within the implantable sensor.

28. The device of claim 25, wherein the processing circuitry detects expansion of the bladder based upon the sensed bladder condition.

29. The device of claim 25, wherein the processing circuitry detects contraction of the bladder based upon the sensed bladder condition.

30. The device of claim 25, wherein the sensor housing includes a vacuum cavity that captures a portion of the exterior surface of the bladder and a fixation element that penetrates the captured portion.

31. The device of claim 25, wherein the implantable sensor includes one or more suture elements to suture the sensor to the exterior surface of the bladder.

32. The system of claim 25, wherein the implantable sensor is sized for at least partial implantation within a wall of the bladder.

33. The system of claim 25, wherein the implantable sensor includes a power supply and a wireless telemetry circuit to transmit the information.
